(19) United States
(12) Plant Patent Application Publication
(10) Pub. No.: US 2002/0174473 P1
(43) Pub. Date: Nov. 21, 2002

(54) Bracteantha plant named Cream

(75) Inventor: Matthew Turner, New South Wales (AU)

Correspondence Address: ROCKEY, MILNAMOW & KATZ, LTD., Two Prudential Plaza, Suite 4700, 180 North Stetson, Chicago, IL, 60601, US

(73) Assignee: Oasis Horticulture PTY, Ltd.

(21) Appl. No.: 09/819,340

(22) Filed: Mar. 28, 2001

(51) Int. Cl. A01H 5/00

(52) U.S. Cl. PLT/359

(57) ABSTRACT

A new and distinct Bracteantha plant named Cream, characterized by its cream colored flowers, upright mounded habit, and medium green leaves.

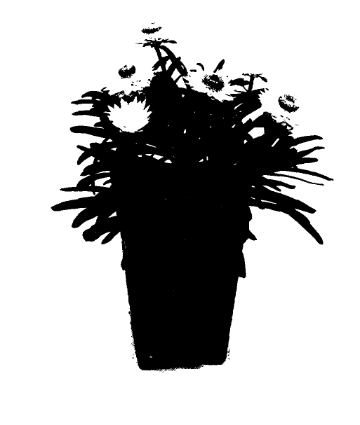

### BACKGROUND OF THE INVENTION

The present invention comprises a new and distinct Bracteantha plant, botanically known as Bracteantha bracteata and hereinafter referred to by the cultivar name Cream.

The new cultivar was developed by the inventor through a controlled breeding program at New South Wales, Australia.

The female (seed) parent of Cream was cultivar Diamond Head (not patented in the United States). The male (pollen) parent of Cream was the proprietary selection designated C-1 (not patented in the United States). Cream was selected as one of the flowering progeny of the above cross.

Asexual reproduction of the new cultivar by terminal tip cuttings has demonstrated that the characteristics of the new cultivar as herein described are firmly fixed and retained through successive generations of such asexual propagation.

### SUMMARY OF THE INVENTION

It was found that the cultivar of the present invention:

(a) exhibits cream colored flowers,

(b) forms medium green foliage,

(c) exhibits a good basal branching character and

(d) exhibits an upright growth habit.

When the new cultivar of the present invention is compared to Florabella White, it is found that the new cultivar is more compact, has shorter internodes, different stem and leaf texture and darker flower color as detailed in TABLE A.

### BRIEF DESCRIPTION OF PHOTOGRAPH

The accompanying photograph shows as nearly true as it is reasonably possible to make the same in color illustrations of this type, typical flower and foliage characteristics of the new cultivar. The plants were grown for 12 weeks in a greenhouse at West Chicago, Ill., USA.

### DETAILED BOTANICAL DESCRIPTION

The Cream cultivar has not been observed under all possible environmental conditions to date. Accordingly, it is possible that the phenotype may vary somewhat with variations in the environment, such as temperature, light intensity, and day length.

The chart used in the identification of colors described herein is the R.H.S. Colour Chart of The Royal Horticultural Society, London, England. The color values were determined on Feb. 15, 2001. The readings were taken between 10:00 and 11:00 a.m. under natural daylight conditions. The plants were produced from cuttings taken from stock plants grown in a double polycarbonate covered greenhouse under conditions comparable to those used in commercial practice while utilizing a soilless growth medium and maintaining temperatures of approximately 72° F. during the day and approximately 65° F. during the night. Plants used for the following descriptions and measurements were grown for 14 weeks after the planting of rooted cuttings.

Classification:

Botanical.—Bracteantha bracteata cultivar Cream.

Commercial.—Helichrysum.

Parentage:

Female parent.—Diamond Head.

Male Parent.—Proprietary Section Designated C-1.

Propagation:

Type cutting.—Terminal tip.

Time to Initiate Roots.—Approximately 30 to 50 days with the shorter times generally being experienced in the summer and the longer times in the winter.

Plant description:

Habit of Growth.—Moderately vigorous with good basal branching. Pinching improves basal branching. A mature plant, 14 weeks after the planting of a rooted cutting, commonly measures approximately 15 cm in height, approximately 30 cm in diameter.

Form.—Upright, mounded.

Lateral Stem.—Diameter: Approximately 4 mm. Texture: Hirsute. Color: 145B. Internode length: Approximately 4 mm.

Foliage.—Arrangement: Alternate. Shape: Lanceolate with entire margin, broadly acute apex and clasping base. Size: Approximately 8.8 cm in length and approximately 9 mm at widest point. Upper surface is rough and 137A. Lower surface is smooth and 137C. Venation is 145C.

Flowering description:

Flowering Habit.—Freely flowering.

Natural Flowering Season.—Spring through fall.

Inflorescence type.—Single, composite, persistent.

Bud.—Size: 2 cm in length, 2 cm in diameter.

Inflorescence diameter.—Approximately 4.2 cm.

Ray florets.—Quantity: Approximately 13 whorls. Appearance: Smooth, papery. Shape: Linear. Length: 1.2 cm Width: From 1 to 4 mm.

Capitulum.—Diameter is approximately 1.6 cm, depth is approximately 13 mm.

Disc florets.—Shape: Tubular. Length: Approximately 9 mm.

Flower Color.—Upper and lower surface, fully opened ray floret: 4C with 4A at tip.

Peduncle.—Length: Approximately 8 cm.

Fragrance.—None.

Reproductive Organs.—Stigma color: 21A. Pollen color: 15A.

I claim:

1. A new and distinct cultivar of Bracteantha named Cream substantially as herein shown and described, which:
(a) exhibits cream colored flowers,
(b) forms medium green foliage,
(c) exhibits a good basal branching character and
(d) exhibits an upright mounded and trailing growth habit.
TABLE A

| | | FLORABELLA |
|---|---|---|
| CHARACTERISTIC | CREAM | WHITE |
| PLANT HEIGHT-CM | 15 | 24 |
| PLANT DIAMETER-CM | 30 | 20 |
| # LATERAL BRANCHES | 13 | 11 |
| INTERNODE LENGTH-CM | .5 | 1.5 |
| STEM TEXTURE | HIRSUTE | TOMENTOSE |
| STEM COLOR | 145A | 144B |
| LEAF TEXTURE | PUBERULENT | SMOOTH |
| PEDUNCLE TEXTURE | PUBERULENT | TOMENTOSE |
| RAY FLORET COLOR- | 4C WITH 4A AT TIP | 155A |
| UPPER AND LOWER | | |
| SURFACE | | |
